(19) United States
(12) Patent Application Publication
(10) Pub. No.: US 2006/0100202 A1
(43) Pub. Date: May 11, 2006

(54) Novel compounds

(76) Inventors: Harshad Kantilal Rami, Essex (GB); Mervyn Thompson, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/496,194

(22) PCT Filed: Dec. 19, 2002
(86) PCT No.: PCT/GB02/05812

§ 371 Date: Oct. 18, 2004

(30) Foreign Application Priority Data
Dec. 20, 2001 (GB) 01305550.7

(51) Int. Cl. A61K 31/538; A61K 31/47; A61K 31/405

(52) U.S. Cl. 514/230.5; 544/105; 546/176; 548/495; 514/419; 514/311

(57) ABSTRACT

Compounds of formula (I) or a pharmaceutically acceptable salt or solvate thereof: 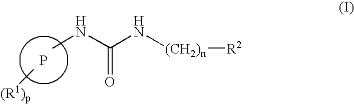
wherein, R¹, R², P, p and n are as defined in the specification, a process for preparing such compounds, a pharmaceutical composition containing such compounds and the use of such compounds in medicine.

This invention relates to novel compounds, especially urea derivatives, having pharmacological activity, processes for their preparation, to compositions containing them and to their use in the treatment of various disorders.

Vanilloids are a class of natural and synthetic compounds which are characterised by the presence of a vanillyl (3-hydroxy 4-methoxyphenyl) group or a functionally equivalent group. Vanilloid Receptor (VR1), whose function is modulated by such compounds, has been widely studied and is extensively reviewed by Szallasi and Blumberg (The American Society for Pharmacology and Experimental Therapeutics, 1999, Vol. 51, No. 2.).

A wide variety of Vanilloid compounds of different structures are known in the art, for example those disclosed in European Patent Application Numbers EP 0 347 000 and EP 0 401 903, UK Patent Application Number GB 2226313 and International Patent Application, Publication Number WO 92/09285. Particularly notable examples of vanilloid compounds or vanilloid receptor modulators are capsaicin, namely trans 8-methyl-N-vanillyl-6-nonenamide, isolated from the pepper plant, capsazepine (Tetrahedron, Vol. 53, No. 13, pp. 4791- 4814, 1997) and olvanil-N-(3-methoxy-4-hydroxy-benzyl)oleamide (J. Med. Chem. 1993, 36, 2595-2604).

U.S. Pat. No. 3,424,760 and U.S. Pat. No. 3,424,761 both describe a series of 3-Ureidopyrrolidines that are said to exhibit analgesic, central nervous system, and pyschopharmacologic activities. These patents specifically disclose the compounds 1-(1-phenyl-3-pyrrolidinyl)-3-phenyl urea and 1-(1-phenyl-3-pyrrolidinyl)-3-(4-methoxyphenyl)urea respectively.

International Patent Applications, Publication Numbers WO 02/08221, WO 02/16317, WO 02/16318 and WO 02/16319 each disclose certain vanilloid receptor antagonists and their use in the treatment of diseases associated with the activity of the vanilloid receptor.

Co-pending International Patent Application Number PCT/EP02/04802 discloses a series of urea derivatives and their use in the treatment of diseases associated with the activity of the vanilloid receptor.

A structurally novel class of compounds has now been found which also possess Vanilloid receptor (VR1) antagonist activity. The present invention therefore provides, in a first aspect, a compound of formula (I) or a pharmaceutically acceptable salt or solvate thereof: 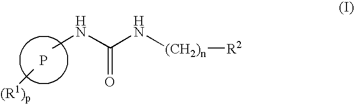
wherein,
P is phenyl, naphthyl or heterocyclyl;
R¹ is selected from —H, halo, alkyl, alkoxy, cycloalkyl, aralkyl, aralkoxy, cycloalkylalkyl, cycloalkylalkoxy, —CN, —NO₂, —OH, —OCF₃, —CF₃, —NR⁵R⁶, —S(O)ₘR⁷, —S(O)₂NR⁵R⁶, —OS(O)₂R⁷, —OS(O)₂CF₃, —O(CH₂)ₓNR⁵R⁶, —C(O)CF₃, —C(O)alkyl, —C(O)cycloalkyl, —C(O)aralkyl, —C(O)Ar, —C(O)(CH₂)ₓOR⁷, —C(O)(CH₂)ₓNR⁵R⁶, —C(O)alkoxy, —C(O)NR⁵R⁶, —(CH₂)ₓC(O)alkoxy, —(CH₂)ₓOC(O)R⁷, —(CH₂)ₓOR⁷, —(CH₂)ₓR⁵R⁶, —(CH₂)ₓC(O)NR⁵R⁶, —(CH₂)ₓN(R⁵)C(O)R⁷, —(CH₂)ₓS(O)₂NR⁵R⁶, —(CH₂)ₓN(R⁵)S(O)₂R⁷, -ZAr, —(CH₂)ₓS(O)₂R⁷, —(OCH₂)ₓS(O)₂R⁷, —N(R⁵)S(O)₂R⁷, —N(R⁵)C(O)R⁷, —(CH₂)ₓN(R⁵)S(O)₂R⁷, —(CH₂)ₓN(R⁵)C(O)R⁷ or —(CH₂)ₓC(O)alkyl;
R² is the group: 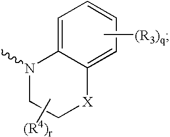
X is a bond, C, O or NR⁸;
R³ is selected from —H, halo, alkyl, alkoxy, cycloalkyl, aryl, aralkyl, aralkoxy, cycloalkylalkyl, cycloalkylalkoxy, —CN, —NO₂, —OH, —OCF₃, —CF₃, —NR⁵R⁶, —S(O)ₘR⁷, —S(O)₂NR⁵R⁶, —OS(O)₂R⁷, —OS(O)₂CF₃, —O(CH₂)ₓNR⁵R⁶, —C(O)CF₃, —C(O)alkyl, —C(O)cycloalkyl, —C(O)aralkyl, —C(O)Ar, —C(O)(CH₂)ₓOR⁷, —C(O)(CH₂)ₓNR⁵R⁶, —C(O)alkoxy, —C(O)NR⁵R⁶, —(CH₂)ₓC(O)alkoxy, —(CH₂)ₓOC(O)R⁷, —(CH₂)ₓOR⁷, —(CH₂)ₓR⁵R⁶, —(CH₂)ₓC(O)NR⁵R⁶, —(CH₂)ₓN(R⁵)C(O)R⁷, —(CH₂)ₓS(O)₂NR⁵R⁶, —(CH₂)ₓN(R⁵)S(O)₂R⁷, -ZAr, —(CH₂)ₓS(O)₂R⁷, —(OCH₂)ₓS(O)₂R⁷, —N(R⁵)S(O)₂R⁷, —N(R⁵)C(O)R⁷, —(CH₂)ₓN(R⁵)S(O)₂R⁷, —(CH₂)ₓN(R⁵)C(O)R⁷ or —(CH₂)ₓC(O)alkyl;
R⁴ is hydrogen or alkyl;
R⁵ and R⁶ may be the same or different and represent H or alkyl or R⁵ and R⁶ together with the atoms to which they are attached form a C₃₋₆azacycloalkane, C₃₋₆(2-oxo)azacycloalkane ring or C₅₋₈ polymethylene chain optionally interrupted by heteroatoms such as O or —NR⁸;
Z is O, S or NR⁸;
R⁷ is alkyl or aryl;
R⁸ is hydrogen, alkyl or aryl;
n is 2, 3, 4, 5 or 6;
p is 0, 1, 2, 3 or 4;
q is 0, 1, 2 or 3;
r is 0, 1 or 2; and
x is 0, 1, 2, 3, 4, 5 or 6.

Suitably, P is phenyl, naphthyl, cinnolinyl or isoquinolinyl. When P is naphthyl, a preferred group is naphth-1-yl. Preferably, P is phenyl.

Suitably, R¹ is halo, alkyl, alkoxy, —C(O)alkyl, —NO₂, —CF₃, —CN or —OCF₃. More suitably, R¹ is halo, alkyl, —C(O)alkyl or —OCF₃. Preferably, R¹ is halo, —C(O)Me or —OCF₃.

Suitably, R² is 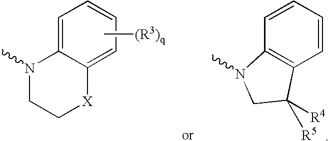
Preferably, R² is dihydroindolyl, tetrahydroydroquinolinyl or dihydrobenzo[1,4]oxazinyl. Most preferably, R² is 2,3-dihydroindol-1-yl, 3,4-dihydro-2H-quinolin-1-yl or 2,3-dihydrobenzo[1,4]oxazin-4-yl.

Suitably, R³ is halo, alkyl, alkoxy, —CF₃, —CN or aryl. More suitably, R³ is halo or alkyl. Preferably, R³ is fluoro or methyl. Most preferably, R³ is a methyl or fluoro substituted at either the 4- or 5-position on the dihydroindole ring, a methyl group substituted on the 6-position of the dihydroquinolinyl ring or a methyl group substituted on the 7-position of the dihydrobenzo[1,4]oxazinyl ring.

Suitably, R⁴ is alkyl. Preferably, R⁴ is methyl.

Suitably, R⁵ is alkyl. Preferably, R⁵ is methyl.

When p is 2 or 3 the groups R¹ may be the same or different. Preferably, p is 1 or 2.

When r is 2 the groups R⁴ may be the same or different. Preferably, r is 0 or 1.

Preferably, n is 2 or 3. Most preferably, n is 2.

Preferably, q is 1 or 2. Most preferably, q is 1.

Preferably, x is O.

Particularly preferred compounds according to the invention include examples E1 to E58 or a pharmaceutically acceptable salt or solvate thereof.

The compounds of the formula (I) can form acid addition salts with acids, such as conventional pharmaceutically acceptable acids, for example maleic, hydrochloric, hydrobromic, phosphoric, acetic, fumaric, salicylic, citric, lactic, mandelic, tartaric and methanesulphonic.

Compounds of formula (I) may also form solvates such as hydrates, and the invention also extends to these forms. When referred to herein, it is understood that the term ‘compound of formula (I)’ also includes these forms.

Certain compounds of formula (I) are capable of existing in stereoisomeric forms including diastereomers and enantiomers and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

As indicated above, the compounds of formula (I) can form salts, especially pharmaceutically acceptable salts. Suitable pharmaceutically acceptable salts are those use conventionally in the art and include those described in J. Pharm. Sci., 1977, 66, 1-19, such as acid addition salts.

Suitable pharmaceutically acceptable salts include acid addition salts.

Suitable pharmaceutically acceptable acid addition salts include salts with inorganic acids such, for example, as hydrochloric acid, hydrobromic acid, orthophosphoric acid or sulphuric acid, or with organic acids such, for example as methanesulphonic acid, toluenesulphonic acid, acetic acid, propionic acid, lactic acid, citric acid, fumaric acid, malic acid, succinic acid, salicylic acid, maleic acid, glycerophosphoric acid or acetylsalicylic acid.

The salts and/or solvates of the compounds of the formula (I) which are not pharmaceutically acceptable may be useful as intermediates in the preparation of pharmaceutically acceptable salts and/or solvates of compounds of formula (I) or the compounds of the formula (I) themselves, and as such form another aspect of the present invention.

The compounds of formula (I) may be prepared in crystalline or non-crystalline form, and if crystalline, may be optionally hydrated or solvated. This invention includes in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

As used herein the term “alkyl” as a group or part of a group refers to a straight or branched chain saturated aliphatic hydrocarbon radical containing 1 to 12 carbon atoms, suitably 1 to 6 carbon atoms. Such alkyl groups in particular include methyl (“Me”), ethyl (“Et”), n-propyl (“Prⁿ”), iso-propyl (“Prⁱ”), n-butyl (“Buⁿ”), sec-butyl (“Bu^{s}”), tert-butyl (“Bu^{t}”), pentyl and hexyl. Where appropriate, such alkyl groups may be substituted by one or more groups selected from halo (such as fluoro, chloro, bromo), —CN, —CF₃, —OH, —OCF₃, C₂₋₆ alkenyl, C₃₋₆ alkynyl, C₁₋₆ alkoxy, aryl and di-C₁₋₆ alkylamino.

As used herein, the term “alkoxy” as a group or part of a group refers to an alkyl ether radical, wherein the term “alkyl” is defined above. Such alkoxy groups in particular include methoxy, ethoxy, n-propoxy, iso-propoxy, n-butoxy, iso-butoxy, sec-butoxy and tert-butoxy. Where appropriate, such alkoxy groups may be substituted by one or more groups selected from halo (such as fluoro, chloro, bromo), —CN, —CF₃, —OH, —OCF₃, C₁₋₆ alkyl, C₂₋₆ alkenyl, C₃₋₆ alkynyl, aryl and di-C₁₋₆ alkylamino.

As used herein, the term “aryl” as a group or part of a group refers to a carbocyclic aromatic radical (“Ar”). Suitably such aryl groups are 5-6 membered monocyclic groups or 8-10 membered fused bicyclic groups, especially phenyl (“Ph”), biphenyl and naphthyl, particularly phenyl.

The term “halo” is used herein to describe, unless otherwise stated, a group selected from fluorine (“fluoro”), chlorine (“chloro”), bromine (“bromo”) or iodine (“iodo”).

The term “naphthyl” is used herein to denote, unless otherwise stated, both naphth-1-yl and naphth-2-yl groups.

The term ‘heterocyclyl’ is used herein to describe, unless otherwise stated, groups comprising one or more rings which may be saturated, unsaturated or aromatic and which may independently contain one or more heteratoms in each ring. Examples of suitable heterocyclyl groups include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolyl, carbazolyl, carbolinyl, chromanyl, chromenyl, cinnolinyl, decahydroquinolinyl, 2H, 6H-1,5,2-dithiazinyl, dihydrobenzofuranyl, furanyl, furazanyl, imidazolyl, 1H-indazolyl, indolinyl, indolyl, isobenzofuranyl, isochromanyl, isoindazolyl, isoindolyl, isoquinolinyl, isothiazolyl, isoxazolyl, naphthyridinyl, oxadiazolyl, 1,2,3-oxadiazolyl, 1,2,4-oxadiazolyl, 1,2,5-oxadiazolyl, 1,3,4-oxadiazolyl, oxazolyl, pyrimidinyl, phthalazinyl, pteridinyl, purinyl, pyrazinyl, pyrazolyl, pyridooxazolyl, pyridoimidazolyl, pyridothiazolyl, pyridyl, pyrrolyl, quinazolinyl, quinolinyl, quinoxalinyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thiazolyl, thienyl, thienothiazolyl, thienooxazolyl, thienoimidazolyl, triazinyl, 1,2,3-triazolyl, 1,2,4-triazolyl, 1,2,5-triazolyl, 1,3,4-triazolyl and xanthenyl.

The present invention also provides, in a further aspect, a process for the preparation of a compound of formula (I) or a pharmaceutically acceptable salt or solvate thereof, which process comprises coupling a compound of formula (II): 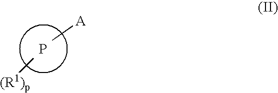
in which R¹, P and p are as defined in formula (I), with a compound of formula (III):
B—(CH₂)ₙ—R² (III)
in which R² and n are as defined in formula (I) and A and B contain the appropriate functional groups which are capable of reacting together to form the urea moiety;
and optionally thereafter if appropriate:
(i) removing any protecting groups;
(ii) forming a pharmaceutically acceptable salt or solvate of the compound so formed.

Suitable examples of appropriate A and B groups include:
(a) A is —N═C═O and B is NH₂; or
(b) A is NH₂ and B is —N═C═O; or
(c) A is NH₂ and B is NH₂ together with an appropriate urea forming agent.

In process (a) or (b) the reaction is carried out in an inert solvent such as dichloromethane or acetonitrile.

In process (c) the urea forming agent can be carbonyl diimidazole or phosgene. The reaction may be performed in an inert organic solvent such as dimethylformamide, tetrahydrofuran or dichloromethane at ambient or elevated temperature. The reaction is typically performed in the presence of a base such as triethylamine or pyridine.

An alternative method of synthesis of the unsymmetrical urea compounds of formula (I) is from a diaryl carbonate, via the corresponding carbamate. Such a methodology is described by Freer et al. (Synthetic Communications, 26(2), 331 - 349, 1996). It will be appreciated that such a methodology could readily be adapted for the preparation of compounds of formula (I).

Those skilled in the art will appreciate that it may be necessary to protect certain groups in the synthesis of compounds of formula (I). Suitable protecting groups and methods for their attachment and removal are conventional in the art of organic chemistry, such as those described in Greene T. W. ‘Protective groups in organic synthesis’ New York, Wiley (1981).

Compounds of formulae (II) and (III) are commercially available or may be prepared according to known methods or analogous to known methods.

Pharmaceutically acceptable salts may be prepared conventionally by reaction with the appropriate acid or acid derivative.

Compounds of formula (I) and their pharmaceutically acceptable salts or solavtes thereof have Vanilloid receptor antagonist (VR1) activity and are believed to be of potential use for the treatment or prophylaxis of certain disorders, or treatment of the pain associated with them, such as: pain, chronic pain, neuropathic pain, postoperative pain, postrheumatoid arthritic pain, osteoarthritic pain, back pain, visceral pain, cancer pain, algesia, neuralgia, dental pain, headache, migraine, neuropathies, carpal tunnel syndrome, diabetic neuropathy, HIV-related neuropathy, post-herpetic neuralgia, fibromyalgia, neuritis, sciatica, nerve injury, ischaemia, neurodegeneration, stroke, post stroke pain, multiple sclerosis, respiratory diseases, asthma, cough, COPD, broncho constriction, inflammatory disorders, oesophagitis, heart burn, Barrett's metaplasia, dysphagia, gastroeosophageal relux disorder (GERD), stomach and duodenal ulcers, functional dyspepsia, irritable bowel syndrome, inflammatory bowel disease, colitis, Crohn's disease, pelvic hypersensitivity, pelvic pain, menstrual pain, renal colic, urinary incontinence, cystitis, burns, itch, psoriasis, pruritis, emesis (hereinafter referred to as the “Disorders of the Invention”).

Thus the invention also provides a compound of formula (I) or a pharmaceutically acceptable salt or solvate thereof, for use as a therapeutic substance, in particular in the treatment or prophylaxis of the Disorders of the Invention.

In particular, the invention provides a compound of formula (I) or a pharmaceutically acceptable salt thereof or a solvate thereof for use in the treatment or prophylaxis of pain and urinary incontinence.

The invention further provides a method of treatment or prophylaxis of the Disorders of the Invention, in mammals including humans, which comprises administering to the sufferer a therapeutically effective amount of a compound of formula (I) or a pharmaceutically acceptable salt or solvate thereof.

The present invention also provides a pharmaceutical composition, which comprises a compound of formula (I) or a pharmaceutically acceptable salt or solvate thereof and a pharmaceutically acceptable carrier.

A pharmaceutical composition of the invention, which may be prepared by admixture, suitably at ambient temperature and atmospheric pressure, is usually adapted for oral, parenteral, rectal administration or intravesical adminstration to the bladder and, as such, may be in the form of tablets, capsules, oral liquid preparations, powders, granules, lozenges, reconstitutable powders, injectable or infusable solutions, suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colourants.

For parenteral administration, fluid unit dosage forms are prepared utilising a compound of the invention or pharmaceutically acceptable salt or solvate thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight, of the active material, depending on the method of administration.

The dose of the compound used in the treatment of the aforementioned disorders will vary in the usual way with the seriousness of the disorders, the weight of the sufferer, and other similar factors. For systemic administration, dosage levels from 0.01 mg to 100 mg per kilogramme of body weight are useful in the treatment of pain. However, as a general guide suitable unit doses may be 0.05 to 1000 mg, more suitably 0.05 to 20, 20 to 250, or 0.1 to 500.0 mg, for example 0.2 to 5 and 0.1 to 250 mg; and such unit doses may be administered more than once a day, for example two or three a day, so that the total daily dosage is in the range of about 0.5 to 1000 mg; and such therapy may extend for a number of weeks or months.

When administered in accordance with the invention, no unacceptable toxicological effects are expected with the compounds of the invention.

The following Examples illustrate the preparation of the compounds of the invention and are not intended to be limiting in any way.

Abbreviations:

AIBN—azoisobutyronitrile
DMF—dimethylformamide
MgSO₄—magnesium sulfate
THF—tetrahydrofuran

### Description 1

### 1-(2-Aminoethyl)indoline (D1)

A mixture of indoline (15 g, 0.126 mol) and 2-bromoethylamine hydrobromide (12.9 g, 0.063 mol) in toluene was heated at reflux for 18 h. After cooling solvent was removed under reduced pressure and the residue dissolved in water. Basification using aqueous potassium carbonate was followed by solvent extraction using dichloromethane. Organic phase was separated, dried over MgSO₄, filtered and concentrated under reduced pressure to leave an oil. Chromatography on silica gel eluting with dichloromethane and methanol (gradient elution, maximum 20%) afforded the title compound as yellow oil (5.45 g, 27%).

### Description 2

### 4-Methylindoline (D2)

To a solution of 4-methylindole (1 g, 7.6 mmol) in glacial acetic acid (10 ml) was added portionwise sodium cyanoborohyride (1.44 g, 0.023 mol) over 15 mins under an argon atmosphere. Stirring was continued for 18 h and water (100 ml) added. Basification using aqueous sodium hydroxide was followed by solvent extraction using dichloromethane. Organic phase was separated, dried (MgSO₄), filtered and concentrated under reduced pressure to leave an oil (0.98 g, 97%).

### Description 3

### 1-(2-Aminoethyl)-4-methylindoline (D3)

The title compound was prepared from 4-methylindoline (D2) using the procedure outlined for Description 1 (0.63 g, 96%).

### Description 4

### 5-Fluoroindoline (D4)

The title compound was prepared from 5-fluoroindole using the procedure outlined for Description 2 (4.1 g, 82%).

### Description 5

### 1-(2-Aminoethyl)-5-fluoroindoline (D5)

The title compound was prepared from 5-fluoroindoline (D4) using the procedure outlined for Description 1 (2.5 g, 92%).

### Description 6

### 5-Methylindoline (D6)

The title compound was prepared from 5-methylindole using the procedure outlined for Description 2 (2.5 g, 50%).

### Description 7

### 1-(2-Aminoethyl)-5-methylindoline (D7)

The title compound was prepared from 5-methylindoline (D6) using the procedure outlined for Description 1 (1.35 g, 82%).

### Description 8

### 4-Fluoroindoline (D8)

The title compound was prepared from 4-fluoroindole using the procedure outlined for Description 2 (1.24 g, 61%).

### Description 9

### 1-(2-Aminoethyl)-4-fluoroindoline (D9)

The title compound was prepared from 4-fluoroindoline (D8) using the procedure outlined for Description 1 (0.7 g, 97%).

### Description 10

### N-(2-Bromo-4-fluorophenyl)acetamide (D10)

To a solution of 2-bromo-4-fluoroaniline (7.35 g, 0.039 mol) and triethylamine (11 ml) in dichloromethane (30 ml) was added a solution of acetyl chloride (2.8 ml) in dichloromethane (20 ml) whilst cooling (ice bath) over a period 20 min. Stirring was continued for 6 h and then dichloromethane partitioned with water. The dichloromethane layer was separated, dried (MgSO₄), filtered and concentrated under reduced pressure to afford the product as a white solid (4.61 g, 49%).

### Description 11

### N-(2-Bromo-4-fluorophenyl)-N-(2-methylallyl)acetamide (D11)

To a cooled solution (ice bath) of (D10) (4.6 g, 0.021 mol) in dry DMF (30 ml) was added sodium hydride (60% dispersion in oil, 0.89 g, 0.022 mol) under an argon atmosphere. After stirring for 10 min 3-bromo-2-methylpropene was added and the reaction stirred at ambient temperature for 18 h. DMF was removed under reduced pressure and the residue partitioned between diethyl ether and water. The organic layer was separated, dried (MgSO₄), filtered and concentrated under reduced pressure to afford an oil. Chromatography on silica gel eluting with diethyl ether afforded the product as a clear oil (5.5 g, 91%).

### Description 12

### 1-(5-Fluoro-3,3-dimethyl-2,3-dihydroindol-1-yl)ethanone (D12)

A solution of (D11) (2.2 g, 8 mmol) in toluene (30 ml) was treated with AIBN (catalytic amount, 30 mg) followed by tributyltin hydride (2.6 ml, 0.01 mol) in toluene (10 ml). Reaction was stirred for 20 min at ambient temperature and then warmed to 50° C. for 90 min. After cooling, the reaction mixture was partitioned with water. The organic layer was separated, dried (MgSO₄), filtered and concentrated under reduced pressure to afford an oil. Chromatography on silica gel eluting with diethyl ether and hexane (gradient elution, maximum 50%), afforded the product as a white solid (0.56 g, 35%).

### Description 13

### 5-Fluoro-3,3-dimethylindoline (D13)

A solution of (D12) (0.78 g, 3.8 mmol) in ethanol (10 ml) and 2M HCl (25 ml) was heated at 85° C. for 2 h and cooled. Basification using sodium hydrogen carbonate was followed by solvent extraction using dichloromethane. Organic phase was separated, dried (MgSO₄), filtered and concentrated under reduced pressure to leave an oil (0.55 g, 87%).

### Description 14

### 2-(5-Fluoro-3,3-dimethyl-2,3-dihydroindol-1-yl)ethylamine (D14)

The title compound was prepared from (D13) using the procedure outlined for Description 1 (0.14 g, 43%).

### Description 15

### 2-(6-Methyl-3,4-dihydro-2H-quinolin-1-yl)ethylamine

The title compound was prepared from 6-methyl-1,2,3,4-tetrahydroquinoline using the procedure outlined for Description 1 (5.85 g, 44%).

### Description 16

### (5-Methyl-2-nitrophenoxy)-acetic acid, ethyl ester (D16)

A solution of 5-methyl-2-nitrophenol (10 g, 0.065 mol) and ethyl bromoacetate (7.25 ml) in acetone (200 ml) containing powdered potassium carbonate (9.91 g) was refluxed for 18 h and cooled. The solid was filtered off and the filtrate concentrated under reduced pressure to afford the product as a light yellow solid (15.22 g, 98%).

### Description 17

### 7-Methyl4H-benzo[1,4]oxazin-3-one (D17)

A solution of (D16) (15.2 g, 0.064 mol) in ethanol (80 ml) and cyclohexene (10 ml) containing 10% palladium on charcoal (0.5 g) was heated at reflux for 27 h and cooled. Catalyst was filtered off and the filtrate concentrated under reduced pressure to afford the compound as a white solid (6.25 g, 60%).

### Description 18

### 7-Methyl-3,4-dihydro-2H-benzo[1,4]oxazine (D18)

A suspension of (D17) (6.0 g, 0.037 mol) in dry THF (50 ml) was treated with borane. THF complex (2.5 eq., 100 ml). The resulting solution was then heated at reflux for 3 h, cooled, basified using aqueous potassium carbonate (10%, 150 ml) and extracted using dichloromethane. Organic phase was separated, dried (MgSO₄), filtered and concentrated under reduced pressure to leave an oil. Chromatography on silica gel eluting with dichloromethane afforded the product as an oil (5.4 g, 98%).

### Description 19

### 2-(7-Methyl-2,3-dihydrobenzo[1,4]oxazin4-yl)ethylamine (D19)

The title compound was prepared from 7-methyl-3,4-dihydro-2H-benzo[1,4]oxazine (D18) using the procedure outlined for Description 1 (2.20 g, 64%).

### Description 20

### (5-Fluoro-2-nitrophenoxy)-acetic acid, ethyl ester (D20)

The title compound was prepared from 5-fluoro-2-nitrophenol using the procedure outlined in Description 16 (15.17 g, 98%).

### Description 21

### 7-Fluoro-4H-benzo[1,4]oxazin-3-one (D21)

The title compound was prepared from (D20) using the procedure outlined for Description 17 (8.15 g, 79%).

### Description 22

### 7-Fluoro-3,4-dihydro-2H-benzo[1,4]oxazine (D22)

The title compound was prepared from (D21) using the procedure outlined for Description 18 (7.15 g, 98%).

### Description 23

### 2-(7-Fluoro-2,3-dihydrobenzo[1,4]oxazin-4-yl)ethylamine (D23)

The title compound was prepared from (D22) using the procedure outlined for Description 19 (2.15 g, 56%).

### EXAMPLE 1

### N-(2-Bromophenyl)-N′-[2-(7-fluoro-2,3-dihydrobenzo[1,4]oxazin-4-yl)ethyl]urea (E1)

To a solution of 2-(7-fluoro-2,3-dihdrobenzo[1,4]oxazin-4-yl)ethylamine (D23) (0.1 g, 0.5 mmol) in dichloromethane (3 ml) was added 2-bromophenyl isocyanate (101 mg, 0.5 mmol) in dichloromethane (2 ml). Reaction was stirred for 18 h and the precipitated solid was filtered off and dried to afford the title compound as a white solid (190 mg, 96%).

¹H NMR (400 MHz, CDCl₃) δ7.96 (d, 1H), 7.51 d, 1H), 7.28 (m, 1H), 6.94 (m, 1H), 6.67 (m, 2H), 6.54 (m, 2H), 4.98 (br, 1H), 4.21 (m, 2H), 3.50 (m, 2H), 3.40 (m, 2H), 3.32 (m, 2H). MH⁺ 394, 396.

The following examples (Table 1) were prepared using a similar procedure to that outlined for E1 with the appropriate amine and isocyanate.

TABLE 1

| Example | MH⁺ |
|---|---|
| N-(4-Fluorophenyl)-N′-[2-(2,3-dihydroindol-1- | 300 |
| yl)ethyl]urea (E2) | |
| N-(3,4-Difluorophenyl)-N′-[2-(2,3-dihydroindol-1- | 318 |
| yl)ethyl]urea (E3) | |
| N-(2-Methyl-3-chlorophenyl)-N′-[2-(2,3-dihydroindol-1- | 330, 332 |
| yl)ethyl]urea (E4) | |
| N-(2,3-Dichlorophenyl)-N′-[2-(2,3-dihydroindol-1- | 314, 316 |
| yl)ethyl]urea (E5) | |
| N-(2-Bromophenyl)-N′-[2-(2,3-dihydroindol-1- | 360, 362 |
| yl)ethyl]urea (E6) | |
| N-(2-(Trifluoromethoxy)phenyl)-N′-[2-(2,3-dihydroindol-1- | 366 |
| yl)ethyl]urea (E7) | |
| N-(2-Iodophenyl)-N′-[2-(2,3-dihydroindol-1- | 408 |
| yl)ethyl]urea (E8) | |
| N-Phenyl-N′-[2-(4-methyl-2,3-dihydroindol-1- | 296 |
| yl)ethyl]urea (E9) | |
| N-(4-Fluorophenyl)-N′-[2-(4-methyl-2,3-dihydroindol-1- | 314 |
| yl)ethyl]urea (E10) | |
| N-(2-Chlorophenyl)-N′-[2-(4-methyl-2,3-dihydroindol-1- | 330, 332 |
| yl)ethyl]urea (E11) | |
| N-(3,4-Difluorophenyl)-N′-[2-(4-methyl-2,3-dihydroindol- | 332 |
| 1-yl)ethyl]urea (E12) | |
| N-(3-Acetylphenyl)-N′-[2-(4-methyl-2,3-dihydroindol-1- | 338 |
| yl)ethyl]urea (E13) | |
| N-(2-Methyl-3-chlorophenyl)-N′-[2-(4-methyl-2,3- | 344, 346 |
| dihydroindol-1-yl)ethyl]urea (E14) | |
| N-(2,3-Dichlorophenyl)-N′-[2-(4-methyl-2,3-dihydroindol-1- | 364, 366 |
| yl)ethyl]urea (E15) | |
| N-(2,5-Dichlorophenyl)-N′-[2-(4-methyl-2,3-dihydroindol-1- | 364, 366 |
| yl)ethyl]urea (E16) | |
| N-(2-Bromophenyl)-N′-[2-(4-methyl-2,3-dihydroindol-1- | 374, 376 |
| yl)ethyl]urea (E17) | |
| N-(2-(Trifluoromethoxy)phenyl)-N′-[2-(4-methyl-2,3- | 380 |
| dihydroindol-1-yl)ethyl]urea (E18) | |
| N-(2-Iodophenyl)-N′-[2-(4-methyl-2,3-dihydroindol-1- | 422 |
| yl)ethyl]urea (E19) | |
| N-(2,5-Dichlorophenyl)-N′-[2-(5-methyl-2,3-dihydroindol-1- | 364, 366 |
| yl)ethyl]urea (E20) | |
| N-(2-Bromophenyl)-N′-[2-(5-methyl-2,3-dihydroindol-1- | 374, 376 |
| yl)ethyl]urea (E21) | |
| N-(4-Fluorophenyl)-N′-[2-(5-methyl-2,3-dihydroindol-1- | 314 |
| yl)ethyl]urea (E22) | |
| N-Phenyl-N′-[2-(4-fluoro-2,3-dihydroindol-1- | 300 |
| yl)ethyl]urea (E23) | |
| N-(4-Fluorophenyl)-N′-[2-(4-fluoro-2,3-dihydroindol-1- | 318 |
| yl)ethyl]urea (E24) | |
| N-(2-Chlorophenyl)-N′-[2-(4-fluoro-2,3-dihydroindol-1- | 334, 336 |
| yl)ethyl]urea (E25) | |
| N-(3,4-Difluorophenyl)-N′-[2-(4-fluoro-2,3-dihydroindol-1- | 336 |
| yl)ethyl]urea (E26) | |
| N-(3-Acetylphenyl)-N′-[2-(4-fluoro-2,3-dihydroindol-1- | 342 |
| yl)ethyl]urea (E27) | |
| N-(2-Methyl-3-chlorophenyl)-N′-[2-(4-fluoro-2,3- | 348, 350 |
| dihydroindol-1-yl)ethyl]urea (E28) | |
| N-(1-Naphthyl)-N′-[2-(4-fluoro-2,3-dihydroindol-1- | 350 |
| yl)ethyl]urea (E29) | |
| N-(2,3-Dichlorophenyl)-N′-[2-(4-fluoro-2,3-dihydroindol-1- | 368, 370 |
| yl)ethyl]urea (E30) | |
| N-(2,5-Dichlorophenyl)-N′-[2-(4-fluoro-2,3-dihydroindol-1- | 368, 370 |
| yl)ethyl]urea (E31) | |
| N-(2-Bromophenyl)-N′-[2-(4-fluoro-2,3-dihydroindol-1- | 378, 380 |
| yl)ethyl]urea (E32) | |
| N-(2-(Trifluoromethoxy)phenyl)-N′-[2-(4-fluoro-2,3- | 383 |
| dihydroindol-1-yl)ethyl]urea (E33) | |
| N-Phenyl-N′-[2-(5-fluoro-2,3-dihydroindol-1- | 300 |
| yl)ethyl]urea (E34) | |
| N-(4-Fluorophenyl)-N′-[2-(5-fluoro-2,3-dihydroindol-1- | 318 |
| yl)ethyl]urea (E35) | |
| N-(2-Chlorophenyl)-N′-[2-(5-fluoro-2,3-dihydroindol-1- | 334, 336 |
| yl)ethyl]urea (E36) | |
| N-(3,4-Difluorophenyl)-N′-[2-(5-fluoro-2,3-dihydroindol-1- | 336 |
| yl)ethyl]urea (E37) | |
| N-(3-Acetylphenyl)-N′-[2-(5-fluoro-2,3-dihydroindol-1- | 342 |
| yl)ethyl]urea (E38) | |
| N-(2-Methyl-3-chlorophenyl)-N′-[2-(5-fluoro-2,3- | 334, 336 |
| dihydroindol-1-yl)ethyl]urea (E39) | |
| N-(1-Naphthyl)-N′-[2-(5-fluoro-2,3-dihydroindol-1- | 350 |
| yl)ethyl]urea (E40) | |
| N-(2,5-Dichlorophenylphenyl)-N′-[2-(5-fluoro-2,3- | 368, 370 |
| dihydroindol-1-yl)ethyl]urea (E41) | |
| N-(2-Bromophenyl)-N′-[2-(5-fluoro-2,3-dihydroindol-1- | 378, 380 |
| yl)ethyl]urea (E42) | |
| N-(2-(Trifluoromethoxy)phenyl)-N′-[2-(5-fluoro-2,3- | 384 |
| dihydroindol-1-yl)ethyl]urea (E43) | |
| N-(2-Iodophenyl)-N′-[2-(5-fluoro-2,3-dihydroindol-1- | 426 |
| yl)ethyl]urea (E44) | |
| N-(2-Bromophenyl)-N′-[2-(6-methyl-3,4-dihydro-2H-quinolin- | 388, 390 |
| 1-yl)ethyl]urea (E45) | |
| N-(4-Fluorophenyl)-N′-[2-(6-methyl-3,4-dihydro-2H-quinolin- | 329 |
| 1-yl)ethyl]urea (E46) | |
| N-(2-Methyl-3-chlorophenyl)-N′-[2-(6-methyl-3,4-dihydro- | 358, 360 |
| 2H-quinolin-1-yl)ethyl]urea (E47) | |
| N-(1-Naphthyl)-N′-[2-(6-methyl-3,4-dihydro-2H-quinolin-1- | 360 |
| yl)ethyl]urea (E48) | |
| N-(2,3-Dichlorophenyl)-N′-[2-(6-methyl-3,4-dihydro-2H- | 378, 380 |
| quinolin-1-yl)ethyl]urea (E49) | |
| N-(2,5-Dichlorophenyl)-N′-[2-(6-methyl-3,4-dihydro-2H- | 378, 380 |
| quinolin-1-yl)ethyl]urea (E50) | |
| N-(4-Fluorophenyl)-N′-[2-(7-methyl-2,3- | 330 |
| dihydrobenzo[1,4]oxazin-4-yl)ethyl]urea (E51) | |
| N-(2-Bromophenyl)-N′-[2-(7-methyl-2,3- | 390, 392 |
| dihydrobenzo[1,4]oxazin-4-yl)ethyl]urea (E52) | |
| N-(4-Fluorophenyl)-N′-[2-(7-fluoro-2,3- | 334 |
| dihydrobenzo[1,4]oxazin-4-yl)ethyl]urea (E53) | |
| N-(2-Bromophenyl)-N′-[2-(5-fluoro-3,3-dimethyl-2,3- | 406, 408 |
| dihydroindol-1-yl)ethyl]urea (E54) | |
| N-(1-Naphthyl)-N′-[2-(5-fluoro-3,3-dimethyl-2,3- | 378 |
| dihydroindol-1-yl)ethyl]urea (E55) | |
| N-(3-Methylcinnolin-5-yl)-N′-[2-(7-fluoro-2,3- | 382 |
| dihydrobenzo[1,4]oxazin-4-yl)ethyl]urea (E56) | |
| N-(1-Methylisoquinolin-5-yl)-N′-[2-(7-fluoro-2,3- | 381 |
| dihydrobenzo[1,4]oxazin-4-yl)ethyl]urea (E57) | |
| N-(3-Methylisoquinolin-5-yl)-N′-[2-(7-fluoro-2,3- | 381 |
| dihydrobenzo[1,4]oxazin-4-yl)ethyl]urea (E58) | |

Pharmacological Data
In Vitro Assay

As referenced above, the compounds of the invention are vanilloid receptor (VR1) antagonists and hence have useful pharmaceutical properties. Vanilloid receptor (VR1) antagonist activity can be confirmed and demonstrated for any particular compound by use of conventional methods, for example those disclosed in standard reference texts such as D. Le Bars, M. Gozarin and S. W. Cadden, Pharmacological Reviews, 2001, 53(4), 597-652] or such other texts mentioned herein.

The screen used for the compounds of this invention was based upon a FLIPR based calcium assay, similar to that described by Smart et al. (British Journal of Pharmacology, 2000, 129, 227-230). Transfected astrocytoma 1321 N1 cells, stably expressing human VR1, were seeded into FLIPR plates at 25,000 cells/well (96-well plate) and cultured overnight.

The cells were subsequently loaded in medium containing 4 μM Fluo-3 AM (Molecular Probes) for 2 hours, at room temperature, in the dark. The plates were then washed 4 times with Tyrode containing 1.5 mM calcium, without probenecid. The cells were pre-incubated with compound or buffer control at room temperature for 30 minutes. Capsaicin (Sigma) was then added to the cells. Compounds having antagonist activity against the human VR1 were identified by detecting differences in fluorescence when measured after capsaicin addition, compared with no compound buffer controls. Thus, for example, in the buffer control capsaicin addition results in an increase in intracellular calcium concentration resulting in fluorescence. A compound having antagonist activity blocks the capsaicin binding to the receptor, there is no signalling and therefore no increase in intracellular calcium levels and consequently lower fluorescence. pKb values are generated from the IC₅₀ values using the Cheng-Prusoff equation.

All compounds tested by the above methodology had pKb>6, preferred compounds [Examples 1, 3, 10, 11, 12, 14-17, 19, 20-25, 27-33, 37, 39-47, 49, 50, 52, 54, 56, 57 and 58] had a pKb>7.0.

What is claimed is:

1. A compound of formula (I) or a pharmaceutically acceptable salt or solvate thereof: 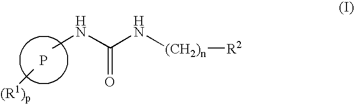
wherein:
P is phenyl, naphthyl or heterocyclyl;
R¹ is selected from —H, halo, alkyl, alkoxy, cycloalkyl, aralkyl, aralkoxy, cycloalkylalkyl, cycloalkylalkoxy, —CN, —NO₂, —OH, —OCF₃, —CF₃, —NR⁵R⁶, —S(O)ₘR⁷, —S(O)₂NR⁵R⁶, —OS(O)₂R⁷, —OS(O)₂CF₃, —O(CH₂)ₓNR⁵R⁶, —C(O)CF₃, —C(O)alkyl, —C(O)cycloalkyl, —C(O)aralkyl, —C(O)Ar, —C(O)(CH₂)ₓOR⁷, —C(O)(CH₂)ₓNR⁵R⁶, —C(O)alkoxy, —C(O)NR⁵R⁶, —(CH₂)ₓC(O)alkoxy, —(CH₂)ₓOC(O)R⁷, —(CH₂)ₓOR⁷, —(CH₂)ₓR⁵R⁶, —(CH₂)ₓC(O)NR⁵R⁶, —(CH₂)ₓN(R⁵)C(O)R⁷, —(CH₂)ₓS(O)₂NR⁵R⁶, —(CH₂)ₓN(R⁵)S(O)₂R⁷, -ZAr, —(CH₂)ₓS(O)₂R⁷, —(OCH₂)ₓS(O)₂R⁷, —N(R⁵)S(O)₂R⁷, —N(R⁵)C(O)R⁷, —(CH₂)ₓN(R⁵)S(O)₂R⁷, —(CH₂)ₓN(R⁵)C(O)R⁷ or —(CH₂)ₓC(O)alkyl;
R² is the group: 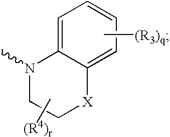
X is a bond, C, O or NR⁸;
R³ is selected from —H, halo, alkyl, alkoxy, cycloalkyl, aryl, aralkyl, aralkoxy, cycloalkylalkyl, cycloalkylalkoxy, —CN, —NO₂, —OH, —OCF₃, —CF₃, —NR⁵R⁶, —S(O)ₘR⁷, —S(O)₂NR⁵R⁶, —OS(O)₂R⁷, —OS(O)₂CF₃, —O(CH₂)ₓNR⁵R⁶, —C(O)CF₃, —C(O)alkyl, —C(O)cycloalkyl, —C(O)aralkyl, —C(O)Ar, —C(O)(CH₂)ₓOR⁷, —C(O)(CH₂)ₓNR⁵R⁶, —C(O)alkoxy, —C(O)NR⁵R⁶, —(CH₂)ₓC(O)alkoxy, —(CH₂)ₓOC(O) R⁷, —(CH₂)ₓOR⁷, —(CH₂)ₓR⁵R⁶, —(CH₂)ₓC(O)NR⁵R⁶, —(CH₂)ₓN(R⁵)C(O)R⁷, —(CH₂)ₓS(O)₂NR⁵R⁶, —(CH₂)ₓN(R⁵)S(O)₂R⁷, -ZAr, —(CH₂)ₓS(O)₂R⁷, —(OCH₂)ₓS(O)₂R⁷, —N(R⁵)S(O)₂R⁷, —N(R⁵)C(O)R⁷, —(CH₂)ₓN(R⁵)S(O)₂R⁷, —(CH₂)ₓN(R⁵)C(O)R⁷ or —(CH₂)ₓC(O)alkyl;
R⁴ is hydrogen or alkyl;
R⁵ and R⁶ may be the same or different and represent H or alkyl or R⁵ and
R⁶ together with the atoms to which they are attached form a C₃₋₆azacycloalkane, C₃₋₆(2-oxo)azacycloalkane ring or C₅₋₈ polymethylene chain optionally interrupted by heteroatoms such as O or —NR⁸;
Z is O, S or NR⁸;
R⁷ is alkyl or aryl;
R⁸ is hydrogen, alkyl or aryl;
n is 2, 3, 4, 5 or 6;
p is 0, 1, 2, 3 or 4;
q is 0, 1, 2 or 3;
r is 0, 1 or 2; and
x is 0, 1, 2, 3, 4, 5 or 6.

2. A compound of formula (I) as claimed in claim 1, in which P is phenyl.

3. A compound of formula (I) as claimed in claim 1, in which n is 2.

4. A compound of formula (I) as claimed in claim 1, in which R² is

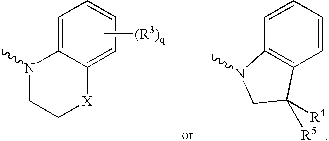

5. A compound of formula (I) as claimed in claim 4, in which R² is dihydroindolyl, tetrahydroydroquinolinyl or dihydrobenzo[1,4]oxazinyl.

6. A compound according to claim 1 in which R³ is halo or alkyl.

7. A compound according to claim 1 which is compound Example number E1 to E58 or a pharmaceutically acceptable salt or solvate thereof.

8. A process for the preparation of a compound of formula (I) or a pharmaceutically acceptable salt or solvate thereof, which process comprises coupling a compound of formula (II): 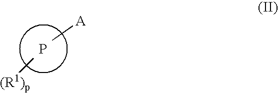
in which R¹, P and p are as defined in formula (I) with a compound of formula (III):
B—(CH₂)ₙ—R² (III)
in which R² and n are as defined in formula (I) and A and B contain the appropriate functional groups which are capable of reacting together to form the urea moiety;
and optionally thereafter if appropriate:
(i) removing any protecting groups;
(ii) forming a pharmaceutically acceptable salt or solvate of the compound so formed.

9. A compound of formula (I), as claimed in claim 1 for use in therapy.

10. A pharmaceutical composition which comprises a compound of formula (I) as claimed in claim 1 and a pharmaceutically acceptable carrier or excipient.

11. The use of a compound of formula (I) as claimed in claim 1, or a pharmaceutically acceptable salt or solvate thereof, in the manufacture of a medicament for the treatment or prophylaxis of disorders in which an antagonist of the vanilloid receptor (VR1) is beneficial.

12. A method of treating disorders or diseases in which an antagonist of the vanilloid receptor (VR1) is beneficial which comprises administering a safe and therapeutically effective amount to a patient in need thereof of a compound of formula (I) as claimed in claim 1, or a pharmaceutically effective salt or solvate thereof.
